(19) United States
(12) Patent Application Publication
(10) Pub. No.: US 2025/0169725 A1
(43) Pub. Date: May 29, 2025

(54) EXPONENTIAL SIGNAL MEASUREMENT DEVICE

(71) Applicant: Worcester Polytechnic Institute, Worcester, MA (US)

(72) Inventors: Ian Costanzo, Worceseter, MA (US); Devdip Sen, Worcester, MA (US); Ulkuhan Guler, Shrewsbury, MA (US); John A. McNeill, Worcester, MA (US)

(21) Appl. No.: 18/511,573

(22) Filed: Nov. 16, 2023

Related U.S. Application Data
(60) Provisional application No. 63/413,742, filed on Oct. 6, 2022.

(51) Int. Cl. A61B 5/1455

(52) U.S. Cl. CPC A61B 5/14552; A61B 2562/0233

(57) ABSTRACT

A nonuniform sampling device and technique and an offset immune luminescent lifetime estimation provides for sensing a gaseous presence and magnitude such as O₂ levels or concentration. The partial pressure of O₂ closely correlates with the lifetime of the luminescence quenched by O₂. Lifetime measurements are preferred over intensity measurements because of their robustness to factors such as variations in the optical path. The disclosed approach, implemented in 180 nm CMOS, features a specialized AFE (Analog Front End) that obtains the luminescent lifetime information with 1) low noise and 2) leverages time-gated excitation, removing the need for optical filters to extract the luminescent response from the excitation light. The lifetime (τ) is calculated using the time differences between equal voltage steps and 4) a measured mean error is as accurate as 0.3%.

### RELATED APPLICATIONS

This patent application claims the benefit under 35 U.S.C. § 119(e) of U.S. Provisional Patent App. No. 63/413,742, filed Oct. 6, 2022, entitled “EXPONENTIAL SIGNAL MEASUREMENT DEVICE,” incorporated herein by reference in entirety.

### BACKGROUND

Among the vital signs of human health, respiratory parameters are key indicators of the physiological condition of the human body. Quantifying the real-time dynamics and physiological distribution of blood gas measurements of oxygen (O2) and carbon dioxide (CO2) is important to clinicians to understand the mechanisms associated with both pathological and normal physiological conditions.

Respiratory health is measured by five major parameters: respiratory rate and quality, arterial partial pressure of oxygen (PaO2), arterial saturation of oxygen (SaO2), and arterial partial pressure of carbon dioxide (PaCO2). The respiratory rate refers to the number of times an individual inhales and exhales during a given period. The respiratory quality describes the effectiveness of each breath quantified as vital capacity, maximal respiratory pressure, and expired volume per minute, all of which determine how well individuals inhale and exhale air from their lungs.

### SUMMARY

A nonuniform sampling device and technique and an offset-immune luminescent lifetime estimation provides for sensing a gaseous presence and magnitude such as O₂ levels or concentration. The partial pressure of O₂ closely correlates with the lifetime of the luminescence quenched by O₂. Lifetime measurements are preferred over intensity measurements because of their robustness to factors such as variations in the optical path. The disclosed approach features a specialized AFE (Analog Front End) that obtains the luminescent lifetime information with 1) low noise and 2) leverages time-gated excitation, removing the need for optical filters to extract the luminescent response from the excitation light. The lifetime (τ) is calculated using the time differences between equal voltage steps and a measured mean error is as accurate as 0.3%.

A luminescent film emits a response to a light stimuli of a particular wavelength, and the sensed voltage values provide an exponential response indicative of the gaseous stimuli. The disclosed method of measuring a timing constant in a measurement circuit for voltage pulses receives a set of points or values based on a voltage value from a sensor device, and evaluating a timing exhibited by the set of points taken from the exponential response. An evaluation circuit computes a timing constant based on the evaluated timing, and based on the timing constant, determines a presence and magnitude of a substance sensed by the sensor device.

An example configuration exhibits a nonuniform sampling device and technique for measuring the luminescent lifetime and decay of light-sensitive materials for oxygen sensing. The system features a switched-capacitor circuit to implement fixed-voltage steps for quantization, enabling long integration times without saturating the front-end amplifier. A control circuit automatically tunes the light emitting diode (LED) excitation pulses to avoid overpowering or starving the front end as photodiode current varies with changes in the partial pressure of oxygen (PO₂). Time gating of the front-end integrator removes the need for optical filtering.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of luminescent emissions and decay for oxygen sensing as disclosed herein;

FIGS. 2A-2C show excitation intensity based on the luminescent emissions of FIG. 1;

FIGS. 3A-3C are a graph and circuits for detecting voltage thresholds over which the intensity measurements are taken;

FIG. 4 shows the voltage levels as in FIGS. 3A and 3B shifted for analysis

FIG. 5 shows a circuit diagram for processing the voltage thresholds of FIGS. 2A-4;

FIGS. 6A-6C show example circuit segments in the circuit of FIG. 5;

FIGS. 7A-7C show measured response signals received and/or processed by the circuits of FIGS. 5-6C;

FIG. 8 shows error detection and resolution in the circuits of FIGS. 5-7C; and

FIG. 9A-9B shows graphs of sensed oxygen using the circuits of FIGS. 5-8.

### DETAILED DESCRIPTION

Luminescent gaseous measurements can provide small, localized devices for direct sensing of blood gas levels based on partial pressures from epidermal exposure. In particular, oxygen and carbon dioxide can be detected and a concentration sensed by a skin mounted device having a pulsed LED light emission coupled with a photosensitive device (typically a photodiode) for measuring an emitted decay that varies with the gaseous presence following an LED pulse.

In an example arrangement as disclosed herein, the sensor device is a photodetector and the external stimuli is emitted light from a photoluminescent film (luminescent film). The external stimuli is based on the photoluminescent film in a gaseous environment, and the computed timing constant is indicative of a sensed gas. The evaluation circuit determines a gaseous concentration based on a voltage value resulting from an external stimuli detected by the sensor device. The set of points need only include at least 3 points on an exponential decay driven by the external stimuli, such as an emitted light from the luminescent film excited by an external light pulse. The luminescent film is sensitive to a certain type of gaseous presence, and emits light of a predetermined wavelength in response to a wavelength of the external light pulse. The 3 points lie on a curve defined by the emitted light, and are indicative of the presence and magnitude of the sensed gaseous presence.

For tuning and accuracy, the evaluation circuit may incur several correction factors. The circuit evaluates a plurality of voltage values from the sensor device when the external stimuli is dormant, in the absence of the external light pulse, and adjusts the computed timing constant for a correction factor based on the evaluated plurality of voltage values.

Alternatively, the evaluation circuit identifies a correction factor based on a response slope of voltage values between the voltage values exhibited by the set of points, and adjusts the computed timing constant for the correction factor based on a sensory delay between adjacent points in the set of points.

Any suitable stimuli response may provide the 3 points used in computing the timing constant. Since only the 3 points, and not an extensive iteration of high-precision values is needed, the timing constant may be computed with relatively low cost circuit elements. The correction factors noted above further mitigate any precision loss.

It is therefore apparent that an ability to monitor the respiration of at-risk patients in real-time at home would be significant in preventing respiratory failures. Care providers can assess the effectiveness of respiration by measuring blood gases, namely oxygen (O₂) and carbon dioxide (CO₂). Recently, luminescent gas sensing has reemerged as an attractive method to create miniaturized noninvasive transcutaneous blood gas monitors. Conventional medical devices measure the partial pressure of O₂ and CO₂ molecules diffusing through the skin, directly correlated with arterial O₂ and CO₂. Such conventional approaches often have substantial power and/or size requirements not commensurate with a wearable device. Configurations herein, in contrast, employ a specialized Analog Front End (AFE) with lightweight estimation approaches and computations for accurate sensor readings.

Configurations herein present a nonuniform sampling technique and an offset immune luminescent lifetime estimation algorithm for O₂ sensing. The partial pressure of O₂ (PO₂) closely correlates with the lifetime of the luminescence quenched by O₂. Lifetime measurements are preferred over intensity measurements because of their robustness to factors such as variations in the optical path, reducing the vulnerability to motion artifacts and skin color differences. The luminescent response as affected by the partial O₂ pressure is disclosed in Applicant's previous filing as U.S. application Ser. No. 17/066,570, filed Oct. 9, 2020, entitled “WEARABLE BLOOD GAS MONITOR” and incorporated herein by reference in entirety. The present disclosure provides a circuit for efficient and compact measurement of sampled values using as few as three data points or readings of an optical emission of a predetermined wavelength including but not limited to oxygen sensing based on luminescent film.

Particular configurations of the disclosed system feature a specialized AFE that gathers the luminescent lifetime information, and leverages time-gated excitation, therefore removing the need for optical filters to extract the luminescent response from the excitation light. The lifetime (τ) is calculated with a proposed offset immune calculation using the time differences between equal voltage steps. It is expected that the measured mean error is as accurate as 1.9% without post-processing. Particular configurations demonstrate measured PO₂ using a gas vessel, however other usage contexts and stimuli may be employed. A particular configuration may be affixed to a patient epidermal surface or adjacent other physiological sources for measurement of blood and/or respiration gases.

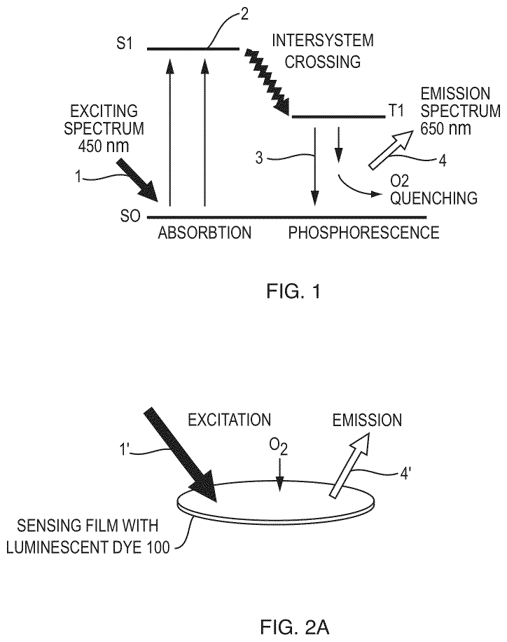

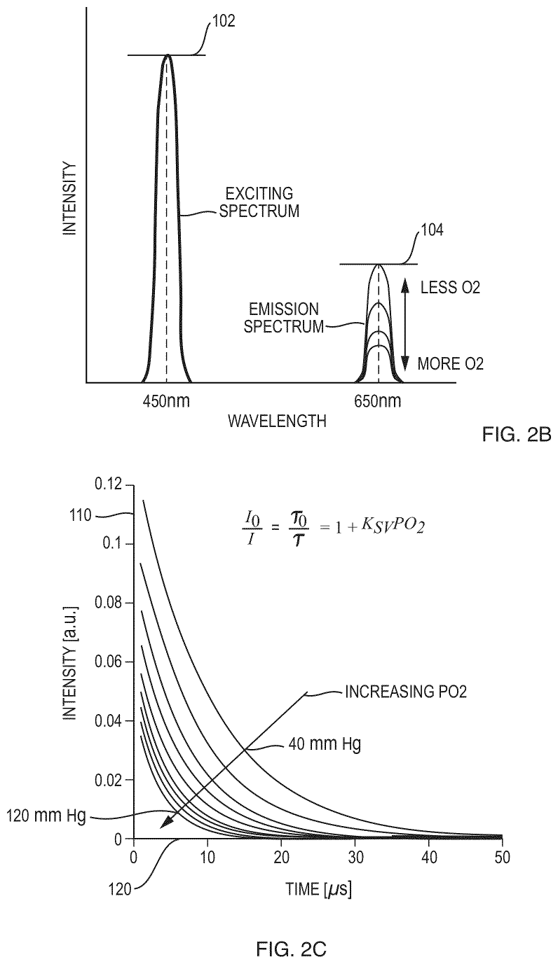

FIG. 1 is a context diagram of luminescent emissions and decay for oxygen sensing as disclosed herein. Referring to FIG. 1, elements of the luminescent O₂ sensing mechanism are illustrated. In the example arrangement, the external stimuli is based on a luminescent film 100 in a gaseous environment, and the computed timing constant is indicative of a sensed gas. An O₂-sensitive dye or film consists of functional groups known as luminophores, including luminescent molecules. Upon irradiation of an LED at a wavelength around 450 nm (blue or violet light), an excited photon interacts with luminescent molecules in the film at step 1. This excites an electron to higher states, at step 2. When the luminophores are exposed to relatively high-energy photons (450 nm light), electrons in the luminophores jump to higher energy levels. After some time, the electrons try to return to the ground state. The electron slowly returns to the ground state, emitting a lower energy photon, as depicted at step 3. For luminescent materials, the excited electrons can enter a triplet state, preventing the electron from returning directly to the ground state. When the electron returns to the ground state from this triplet state, a photon of a longer wavelength is emitted (650 nm). However, O₂ limits or “quenches” emission, as shown at step 4, therefore this emission is “quenched” or suppressed by O₂ present in the sensing area. The dynamics of this interaction are described by the Stern-Volmer equation, shown inside of FIG. 2C. The intensity (I) and τ (timing constant) are related to PO₂ through a rate constant, K_{SV} and intensity/lifetime with no oxygen (I₀/τ₀).

FIGS. 2A-2C show excitation intensity based on the luminescent emissions of FIG. 1. Referring to FIGS. 1-2C, the luminescent molecules are stored or embedded in a sensing film 100. Photoluminescence is one of the possible events resulting from an interaction between light and matter. A molecule capable of luminescence contains functional groups known as luminophores. Luminophores are responsible for the photoluminescent properties of a material. When a luminophore absorbs photons 1′, it is excited to a higher energy state. As the luminophore relaxes from an excited state, it can emit photons in the form of an emission 4′ Typically, when a higher energy (shorter) wavelength enters the material, a lower (longer) energy wavelength is emitted. The difference between these two spectra is known as Stokes's shift.

Two major processes that describe the photon emission during deexcitation are fluorescence and phosphorescence. Fluorescence emission occurs in a wide range of materials, but it is difficult to observe as fluorescent lifetimes are on the order of tens of nanoseconds. The second process is phosphorescence which normally has a lower probability of occurrence, but is easier to observe due to lifetimes on the order of microseconds to seconds. Using a heavy atom in the luminophore improves the probability of phosphorescence and improves the efficiency of the process, reducing the lifetimes.

This photophysical quenching effect can be broadly described as a transfer of energy. A luminescent material, A, is excited by some external energy so that it is elevated to a higher energy state, A*. Then A* is exposed to a quenching species, Q, and some of the energy is transferred to Q by some quenching rate constant, kₛᵥ. The intensity of the excitation 1′ has a peak 102, driven by an LED driver. The resulting emission forms a peak 104, and the intensity of this peak varies with the O₂ presence, where the greater the intensity 104 the lower the oxygen presence, since the oxygen has a tendency to quench the emission 4′. FIG. 2C shows intensity 110 over time 120, where the decay of the intensity 110 over time 120 gives the oxygen concentration.

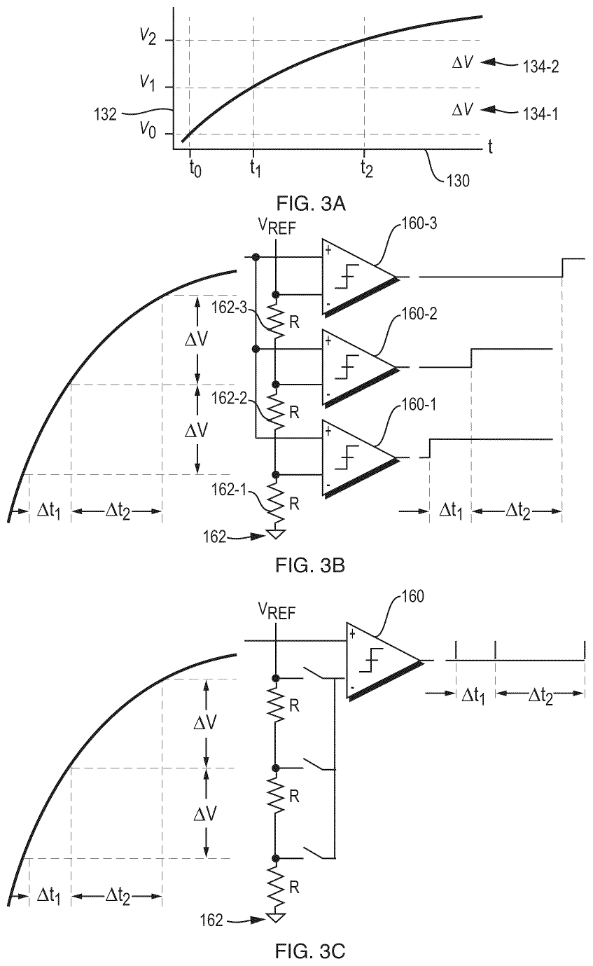

FIGS. 3A-3C show a graph and circuit for detecting voltage thresholds over which the intensity measurements are taken. Referring to FIGS. 1-3C, intensity signal gathering is shown. The nonuniform luminescent lifetime estimation computation commences using a decaying exponential with an offset:

$$i ( t ) = {i}_{pk} exp ( - \frac{t}{τ} ) + {i}_{dc}$$

Using an integrator circuit or similar computational element gives a voltage at a time t:

$$V ( t ) = \frac{1}{C} \int {i}_{pk} exp ( - \frac{t}{τ} ) + {i}_{dc} dt$$

The integration operation scales and inverts the exponential but it does not affect the timing constant. We now have a voltage signal that we can quantize:

$$V ( t ) = \frac{- {i}_{pk} τ}{C} exp ( - \frac{t}{τ} ) + \frac{{i}_{dc} t}{C} + constant$$

Let the integration proceed in time 130, recording the time it takes for the voltage 132 to increase by a fixed amount ΔV. The disclosed circuit need only measure three samples, one at an arbitrary point in time t₀ and the rest at a subsequent voltage step 134-1 . . . 134-2 of equal magnitude, as illustrated in FIG. 3A. The samples define the set of points based on a voltage value, and only 3 are needed to compute the corresponding timing constant. The set of points are taken from t₀, t₁ and t₂, and based on equal voltage intervals ΔV.

We now have three voltages separated by fixed ΔV s. For notation simplicity, we negative sign may be dropped from the amplitude component.

$${V}_{0} = \frac{1}{C} {\int }_{0}^{{t}_{0}} {i}_{d c} + {i}_{p k} exp ( \frac{− t}{τ} ) = \frac{{i}_{dc} {t}_{0}}{C} + \frac{{i}_{p k} τ}{C} exp ( \frac{− {t}_{0}}{τ} )$$

$${V}_{1} = {V}_{0} + Δ V = \frac{1}{C} {\int }_{0}^{{t}_{1}} {i}_{{d}_{c}} + {i}_{p k} exp ( \frac{− t}{τ} ) = \frac{{i}_{dc} {t}_{1}}{C} + \frac{{i}_{p k} τ}{C} exp ( \frac{− {t}_{1}}{τ} )$$

$${V}_{2} = {V}_{0} + 2 Δ V = \frac{1}{C} {\int }_{0}^{{t}_{2}} {i}_{d c} + {i}_{p k} exp ( \frac{− t}{τ} ) = \frac{{i}_{dc} {t}_{2}}{C} + \frac{{i}_{p k} τ}{C} exp ( \frac{− {t}_{2}}{τ} )$$

We can take the difference of adjacent voltage to minimize the DC offset component. If the DC current component is small, the time steps relatively close, or the feedback capacitor is relatively large, the DC component drops out.

$${V}_{2} - {V}_{1} = Δ V = \frac{{i}_{p k} τ}{C} ( exp ( - {t}_{2} / τ ) - exp ( - {t}_{2} / τ ) ) +$$

$${V}_{1} - {V}_{0} = Δ V = \frac{{i}_{p k} τ}{C} ( exp ( - {t}_{1} / τ ) - exp ( - {t}_{0} / τ ) ) +$$

Next, we can divide the two voltage differences to cancel out the amplitude of the exponential, leaving just the time information:

$$\frac{Δ V}{Δ V} = \frac{( exp ( \frac{− {t}_{2}}{τ} ) − exp ( \frac{− {t}_{1}}{τ} ) )}{( exp ( \frac{− {t}_{1}}{τ} ) − exp ( \frac{− {t}_{0}}{τ} ) )}$$

Multiple both sides by:

$$exp ( \frac{− {t}_{1}}{τ} ) / exp ( \frac{{t}_{1}}{τ} )$$

Simplifying yields:

$$1 = \frac{( exp ( \frac{{t}_{1} - {t}_{2}}{τ} ) - 1 )}{( 1 - exp ( \frac{{t}_{1} - {t}_{0}}{τ} ) )}$$

The measured time difference may be used to solve for the timing constant τ. The three time values, defining intervals of luminescence decay according to equal sensed voltage values, can be mapped/computed to the oxygen level.

In order to implement the nonuniform luminescent lifetime estimation computation in hardware, components are needed to reliably create repeatable voltage steps for computing the level crossings at each voltage step. The level crossing detector can be implemented as a resistor string 162-1 . . . 162-3 (162 generally) across several comparators as shown in FIG. 3B. As the integrated voltage rises, each of 3 comparators 160-1 . . . 160-3 (160 generally) trips at a set voltage level corresponding to the voltage steps 134-1, 134-2 and at the recorded times t₀-t₂. This technique captures the concept of the nonuniform sampling but it is exposed to several issues. The technique requires three comparators which can present challenges in power, component cost and size. The three comparators and resistors are matched to ensure the voltage steps 134 are equal.

Matching and power issues in the comparator can be solved by having some additional logic that changes the point on the resistor string 162 that the comparators 160 reference. This technique, shown in FIG. 3C, needs only a single comparator 160. It still may be sensitive to resistor matching and noise, though these can be mitigated with shuffling and averaging. This technique is suited to a particular dynamic range, as the integrated voltage swings between ground and the reference voltage. The resistor string allows for receiving the voltage values from a set of interconnected comparators, and/or for receiving the voltage values from a single comparator adapted to sense a current level based on three sensed voltage levels. Additional voltage levels/thresholds may be employed with corresponding comparators and voltage thresholds.

Invoking the level crossing detectors of FIGS. 3B and 3C, as applied to an example configuration, an intensity sensor, defined by a photodiode, measures an intensity signal having a magnitude which varies with the intensity of the emission 4′. In the example configuration, using a photodiode for sensing the emission 4′, a current flow through the photodiode varies with the emission intensity. The photodiode may have an integrated red-pass filter. The disclosed method of measuring a timing constant in a measurement circuit for voltage pulses includes receiving a set of points based on a voltage value from a sensor device, and evaluating a timing exhibited by the set of points. The AFE computes the timing constant based on the evaluated timing, and based on the timing constant, determines a presence and magnitude of a substance sensed by the sensor device, here O₂. The computed gaseous concentration is based on a voltage value and/or current level resulting from an external stimuli presented by the intensity of the emission 4′ and detected by the sensor device. It should be recognized that the level crossing exhibited as a voltage level may also be implemented with current sensing by manipulating the dependency of current, voltage and resistance accordingly.

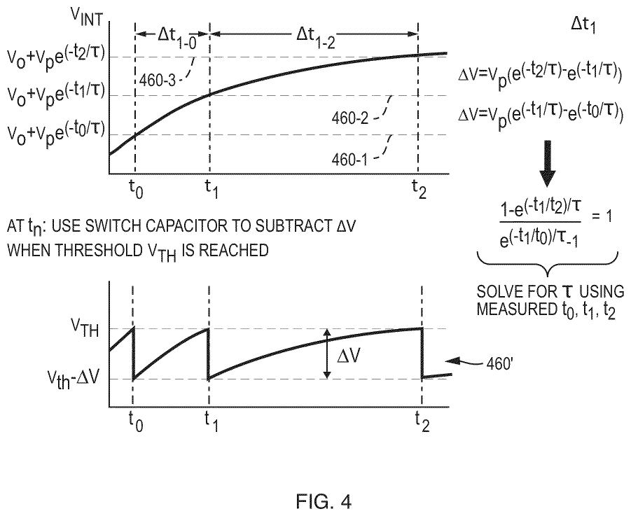

FIG. 4 shows the voltage levels as in FIGS. 3A and 3B shifted for analysis over the same computed timing thresholds t₀-t₂. in the proposed luminescence lifetime estimation approach. The AFE uses the time differences (Δt) between fixed-voltage steps (ΔV) 460-1 . . . 460-3) (460 generally) to extract the timing constant (τ) of the decaying exponential. This computation, implementable in hardware, is a modification of rapid lifetime determination. The nonuniform sampling circuit may include a level crossing circuit for computing a time based on a duration at which the decaying detection signal attains a predetermined voltage demarcated by an equal voltage threshold. A level shifting circuit isolates a duration of each of the threshold crossings based on received voltage values corresponding to the decaying detection signal.

Each voltage step 460 corresponds to resistance values set by the resistor string 162 for activating a corresponding comparator 160. The integration of an exponential does not change the timing constant (τ), the key parameter sought for measurement. Integrating the current generated by the photodiode captures the exponential signal. When the integrated signal voltage reaches a pre-set threshold, the system records the time of the level crossing (e.g., t₀, t₁), and the integration proceeds without interruption. Taking the difference between two level crossings eliminates the integration constant. Determination of the level crossings at t₀-t₂ effectively shifts the voltage steps 460 to a single voltage range 460′. This operation removes any offset errors in the system. The quotient of two differences removes the amplitude component of the exponential. The result is a transcendental equation that is only dependent on the level crossings' sample times, t₀, t₁, and t₂, and the unknown luminescent timing constant, τ. A basic root-finding algorithm calculates t within a few iterations. This lightweight, offset immune algorithm requires only three samples, relaxing data rate requirements.

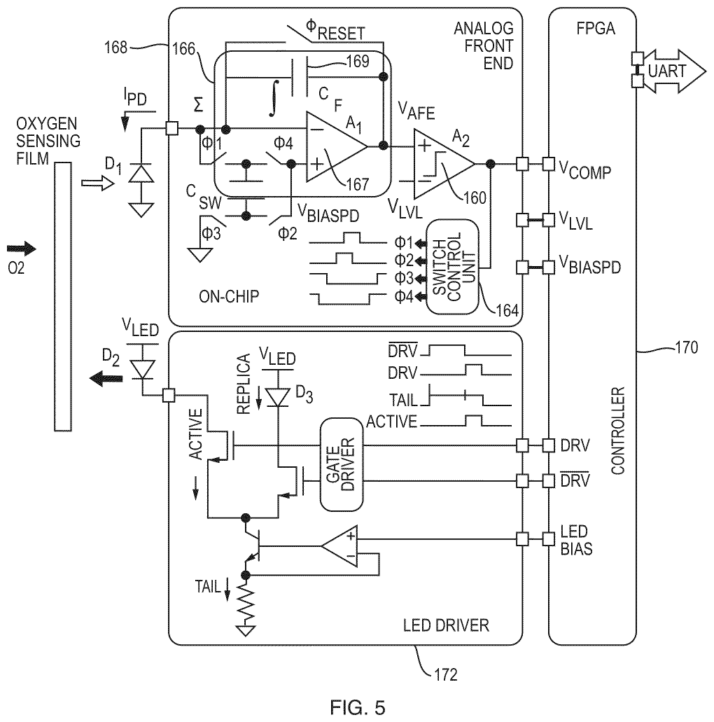

FIG. 5 shows a circuit diagram for processing the voltage thresholds of FIGS. 2A-4. In FIG. 5, circuit for evaluating an exponential decay is presented, including the sensor device such as a photodiode for sending a detection signal indicative of an exponentially decaying stimuli, and a stimulation circuit for generating a triggering signal (excitation signal 1′) for triggering the exponentially decaying stimuli (emission 4′). A nonuniform sampling circuit computes a timing constant defining a rate of decay of the exponentially decaying stimuli, and a results estimator maps the timing constant to a concentration level of a measured substance, where the exponentially decaying stimuli is indicative of the measured substance, such as O₂. A similar circuit may be employed for other stimuli using evaluation of the exponentially decaying stimuli/response for any suitable medium.

Referring to FIG. 5, and continuing to refer to FIGS. 1-4, operation is divided into an excitation phase and a readout phase. During the excitation phase, the light-emitting diode (LED) driver creates a pulse at a set intensity and duration, exciting the O₂-sensitive film with blue light (λ=450 nm). The integrator, A₁ and C_{F}, is set to unity-gain with a switch (φ_{RESET}) to avoid saturating the AFE with the high-intensity LED burst. C_{F} 169 is only reset during the excitation phase, minimizing the impact of reset noise. In addition, the time-domain signal gating via φ_{RESET} removes the need for optical filtering to extract the relatively weak luminescent signal from the powerful excitation signal.

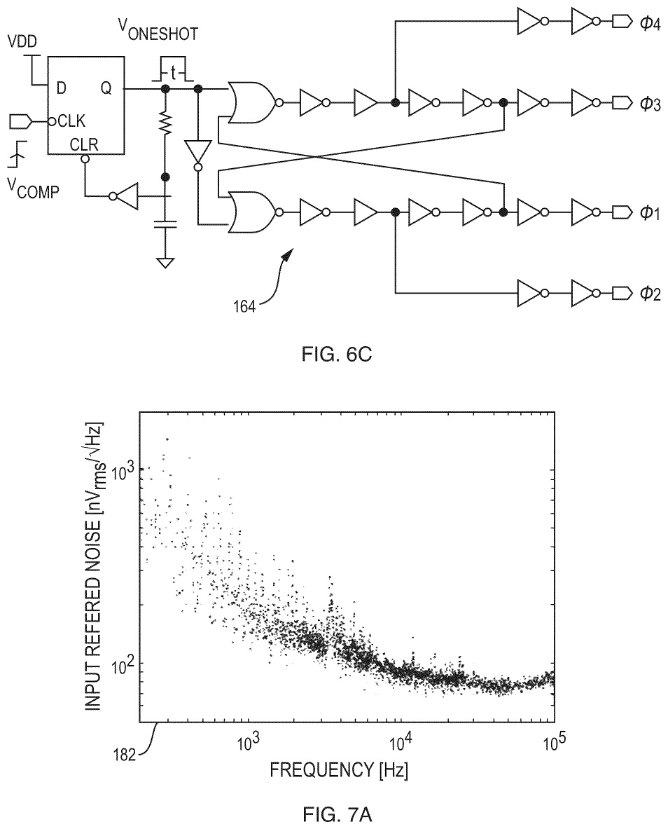

In the readout phase, a photodiode, D₁, captures the emitted red photons (λ=650 nm), generating a current (I_{P D}) that matches the decay of the photons, depicted below in FIG. 7C. A₁ 167 and C_{F} 169 integrate this current to generate a voltage. The integrator is assisted with a nonuniform switched-capacitor technique to improve the dynamic range of its output, V_{AFE}. The comparator, A₂, (160) detects the level crossings and sends a signal (V_{COMP}) to the switch control unit (SCU) 164. The SCU in turn sends four non-overlapping signals (φ₁-φ₄) to drive the switched capacitor, C_{SW}. This action injects a fixed amount of charge into the summing node of the integrator, Σ 166. The charge injection causes a fixed voltage subtraction, ΔV, at V_{AFE}, allowing A₁ to continue integrating the photocurrent.

In a particular configuration, there may be a comparator 160 delay related to the overdrive voltage (Vₒᵥ), how much the input signal exceeds the comparator threshold value. The more the input exceeds the threshold the easier it is for the comparator to make a decision. Steeper rise times earlier on exponential will cross the threshold faster, resulting in a larger overdrive voltage during the decision time of the comparator. Shallower rise times later on the exponential take longer to cross the threshold and thus the comparator takes longer to make a decision.

The device and circuit may evaluate a plurality of voltage values from the sensor device when the external stimuli is dormant, and adjust the computed timing constant for a correction factor based on the evaluated plurality of voltage values.

The digital logic may be implemented on a suitable FPGA (Field Programmable Gate Array) or other processing controller, which controls power-up sequence, excitation and readout phase timing, front-end bias, and LED driver power. A significant role of the FPGA 170 was the automatic “gain control” for the LED driver. The FPGA 170 counts the number of comparator 160 (A₂) pulses and adjusts the LED drive 172 to ensure the number of pulses is the same for each measurement. For low PO₂, the luminescent sensor emits many red photons for a given amount of exciting blue photons, so the LED drive current is set weaker. Conversely, the LED drive increases to excite more luminophores for high PO₂. If the number of pulses from the AFE is less than a set threshold, the LED bias increases by one bit or vice versa. If the pulses match the threshold, there is no change. The automatic LED drive tuning expands the O₂ measurement dynamic range.

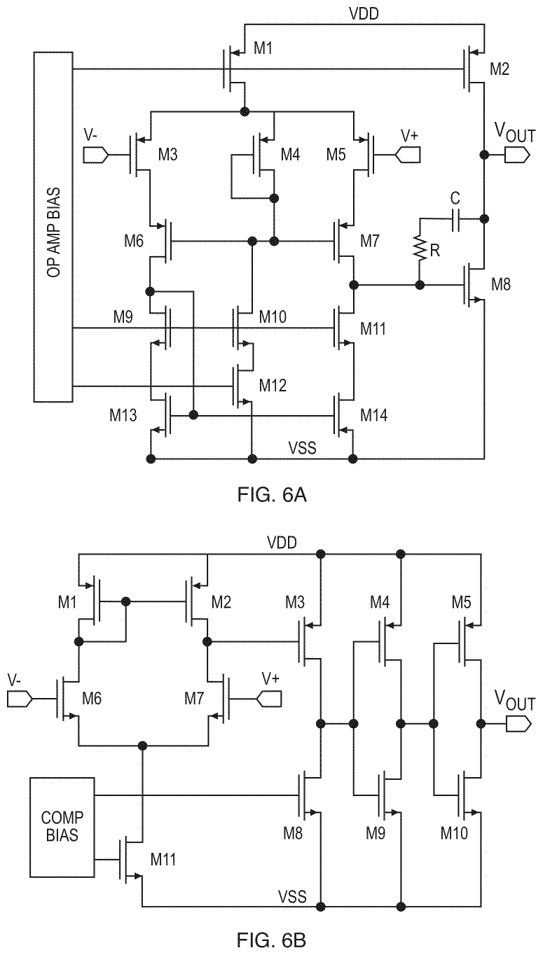

FIGS. 6A-6C show example circuit segments in the circuit of FIG. 5. FIGS. 6A and 6B contain simplified schematics of the AFE 168. Referring to FIGS. 1-6B, the front-end amplifier, A₁ 167 is a telescopic cascode with PMOS inputs with a common source output stage. The timing constant of the front end should be at least one order of magnitude less than the luminescent decay time to reduce the error induced from the front end to less than 1%. The comparator, A₂, performs the level crossing detection function of the sampler. The circuit detects when the output of the integrator reaches the threshold and triggers the switched-capacitor circuit to inject a fixed amount of charge into the summing node of the op-amp and generate the voltage step 460′ (ΔV), depicted in FIG. 4.

To achieve narrow LED pulses, we employed a current steering technique inspired by emitter-coupled logic. A regulated current source controls the tail current that sets the LED drive based on a bias voltage provided by the controller. The controller turns on the replica current path when the main control loop sends the command to excite the O₂ sensing film. This action wakes up the tail current source and establishes the LED drive current. Once the replica path establishes the LED driver current, the controller quickly switches to the active channel to excite the luminophores. The driver can set an LED pulse intensity up to 1 A with 10-bit resolution and pulse width as narrow as a few hundred nanoseconds. FIG. 6C shows an example circuit for the SCU 164.

Graphs described further below and shown in FIGS. 7A-9B illustrate a series of tests, including bandwidth and input-referred noise to evaluate key front end performance; a transient test to assess the hardware implementation of the t-estimation algorithm; and a gas test to demonstrate O₂ measurement performance for the example configuration.

FIGS. 7A-7C show measured response signals received and/or processed by the circuits of FIGS. 5-6C. During a 40 μs excitation phase (30 μs replica, 10 μs active), the auxiliary LED driver uses a maximum of 15 μJ to excite the sensor at high PO₂. During the excitation and readout phase (100 μs), the AFE uses 130 nJ to collect three samples for a t calculation. The input-referred noise 180 and the bandwidth of A₁ are given in FIGS. 7A and 7B, respectively. The integrated noise is 124 μVᵣₘₛ, measured over a bandwidth from 200 Hz to 100 kHz, and the 1/f corner is ˜ 10 kHz. The measured gain-bandwidth product GBWP of the front-end amplifier is 10 MHz.

We calculated the t employing the proposed lifetime estimation algorithm using the time differences between comparator pulses, V_{COMP}. To assess the accuracy and precision of the technique, we fed the AFE with an exponentially decaying current with a controlled timing constant, iₜₑₛₜ in FIG. 7C. The lifetime was also calculated by fitting an exponential model to the same stimulus current waveform for comparison.

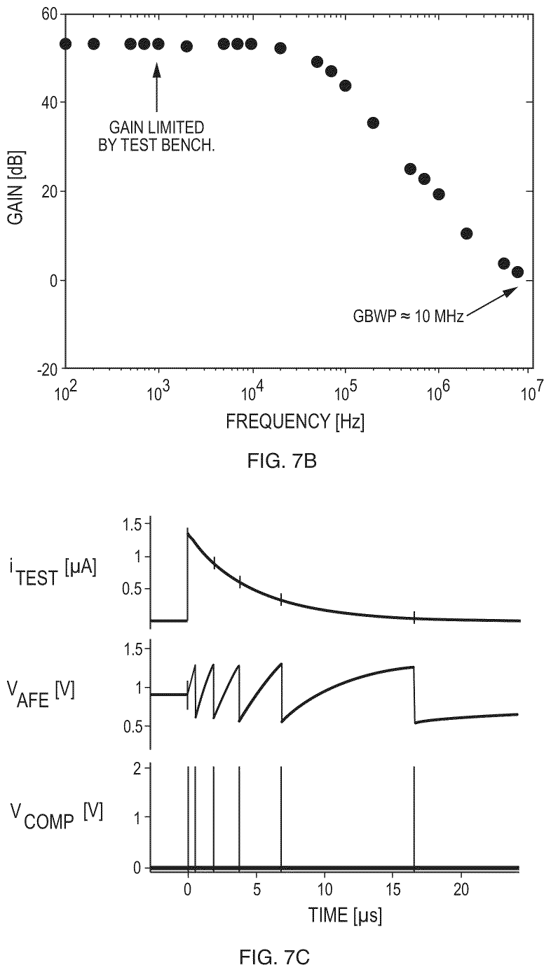

FIG. 8 shows the error between the proposed algorithm and the exponential model. Referring to FIG. 8, for timing constants ranging from 1 to 20 μs, the relevant range for human transcutaneous O₂ estimation, the mean error is ˜−1.9%, with the error bounds +1%/−4%. Increased error for t<1 μs is due to the comparator response delay. With a ΔV of 0.7 V and a C_{F} of 2 pF, the sampler requires a minimum 3 pC to perform a calculation.

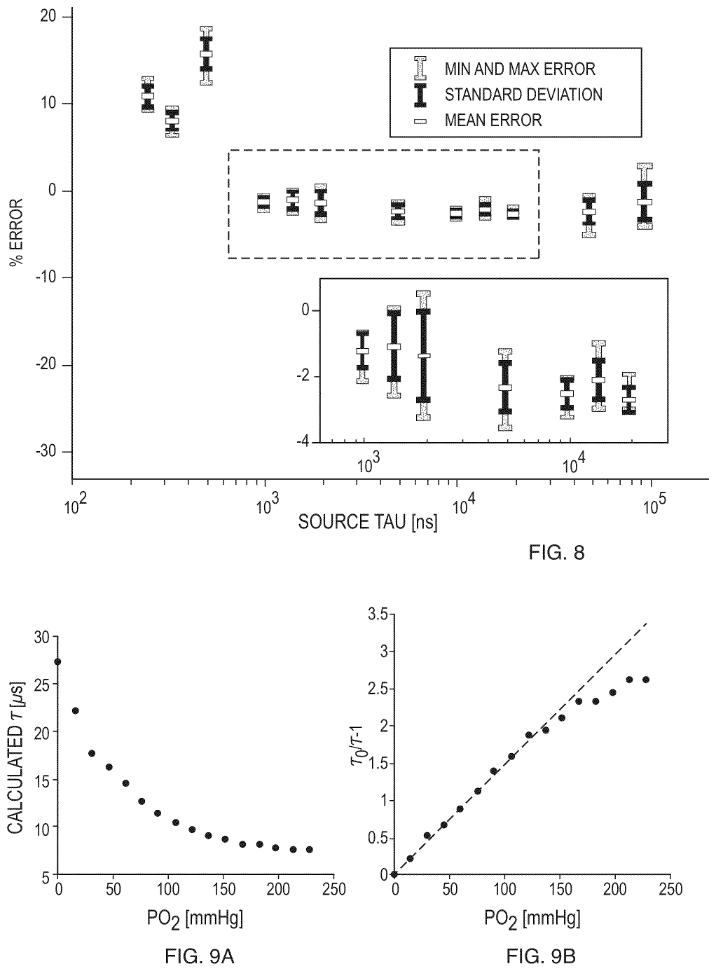

FIG. 9A-9B show graphs of sensed oxygen using the circuits of FIGS. 5-8. An in vitro gas test is a ratiometric mass flow rate use case. An O₂ sensor using the circuit in FIG. 5 is deployed over a range of PO₂. To control the PO₂, we mixed O₂ and nitrogen (N₂) in controlled ratios at 760 mmHg (1 atm). MKS 1179A mass flow controllers (MFC) set the gas mixture ratios. For fixed volume, temperature, and pressure, the partial pressure of a gas is directly related to the mass of the gas in the mixture. The PO₂ was swept from 0 to 228 mmHg in 14.25 mmHg steps. A PC software controls the mass flow set points and the data readout.

The calculated timing constants are plotted against PO₂ in FIGS. 9A and 9B is the Stern-Volmer plot of the same data. The deviation from the linear relationship at relatively high PO₂ is due to some luminophores being inaccessible for quenching by O₂. The nonuniform sampler can measure PO₂ from 0 to 150 mmHg, covering the human-relevant range, with a 2.2 mmHg mean error, lower than the FDA standard of 5 mmHg.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of measuring a time constant in a measurement circuit for voltage pulses, comprising:
receiving a set of points based on a voltage value from a sensor device;
evaluating a timing exhibited by the set of points;
computing a timing constant based on the evaluated timing; and
based on the timing constant, determining a presence and magnitude of a substance sensed by the sensor device.

2. The method of claim 1 further comprising determining a gaseous concentration based on a voltage value resulting from an external stimuli detected by the sensor device.

3. The method of claim 1 wherein the set of points includes at least 3 points on an exponential decay driven by the external stimuli.

4. The method of claim 2 further comprising:
evaluating a plurality of voltage values from the sensor device when the external stimuli is dormant; and
adjusting the computed timing constant for a correction factor based on the evaluated plurality of voltage values.

5. The method of claim 2 further comprising:
identifying a correction factor based on a response slope of voltage values between the voltage values exhibited by the set of points; and
adjusting the computed timing constant for the correction factor based on a sensory delay between adjacent points in the set of points.

6. The method of claim 2 wherein the sensor device is a photodetector and the external stimuli is emitted light.

7. The method of claim 6 wherein the external stimuli is based on a luminescent film in a gaseous environment, and the computed timing constant is indicative of a sensed gas.

8. The method of claim 1 further comprising receiving the voltage values from a set of interconnected comparators.

9. The method of claim 1 further comprising receiving the voltage values from a single comparator adapted to sense a current level based on three sensed voltage levels.

10. The method of claim 9 wherein the received set of points is a respective current value based on a constant voltage difference between the set of points.

11. A circuit for evaluating an exponential decay, comprising:
a sensor device for sending a detection signal indicative of an exponentially decaying stimuli;
a stimulation circuit for generating a triggering signal for triggering the exponentially decaying stimuli;
a nonuniform sampling circuit for computing a timing constant defining a rate of decay of the exponentially decaying stimuli; and
a results estimator for mapping the timing constant to a concentration level of a measured substance, the exponentially decaying stimuli indicative of the measured substance.

12. The circuit of claim 11 wherein the nonuniform sampling circuit includes a level crossing circuit for computing a time based on a duration at which the decaying detection signal attains a predetermined voltage demarcated by an equal voltage threshold.

13. The device of claim 12 further comprising a level shifting circuit for isolating a duration of each of the threshold crossings based on received voltage values corresponding to the decaying detection signal.

14. The device of claim 12 further comprising a resistor string for delivering a timing signal based on a voltage of the detection signal attains the voltage threshold.

15. The device of claim 11 wherein the stimulation circuit is operable to generate the triggering signal at predetermined timing intervals, and the computed time is based on the predetermined timing intervals.

16. The device of claim 15 wherein the triggering signal is an optical signal of a predetermined wavelength and the decaying stimuli is a luminescent decay based on an emitted wavelength quenched by a presence of the measured substance.

17. The device of claim 13 wherein the level shifting circuit is further configured for correcting a DC leakage current by
evaluating a plurality of voltage values from the sensor device when the exponentially decaying stimuli is dormant; and
adjusting the computed timing constant for a correction factor based on the evaluated plurality of voltage values.

18. The device of claim 13 wherein the level shifting circuit is further configured for:
identifying a correction factor based on a response slope of voltage values between the voltage values exhibited by the received voltage values; and
adjusting the computed timing constant for the correction factor based on a sensory delay between received voltage values.
